(19) United States
(12) Patent Application Publication
(10) Pub. No.: US 2012/0219591 A1
(43) Pub. Date: Aug. 30, 2012

(54) USE OF VECTORS EXPRESSING INTRACELLULAR POLYNUCLEOTIDE BINDING PROTEINS AS ADJUVANTS

(76) Inventors: Karl Ljungberg, Stockholm (SE); Alvaro Lladser, San Miguel (CL); Rolf Kiessling, Stockholm (SE)

(21) Appl. No.: 13/138,869

(22) PCT Filed: Oct. 22, 2009
(86) PCT No.: PCT/US09/05753

§ 371 Date: Mar. 27, 2012

Related U.S. Application Data
(60) Provisional application No. 61/212,554, filed on Apr. 13, 2009.

(51) Int. Cl. A61K 39/00; A61P 37/04

(52) U.S. Cl. 424/278.1

(57) ABSTRACT

An object of the invention is to provide a vaccine adjuvant that is comprised of a vector capable of expressing a polynucleotide binding protein in excess of levels already present in cells. Expression of the polynucleotide binding protein initiates intracellular pathways that lead to expression of type 1 interferons. In concert with expression of type 1 interferons, other cytokines are expressed, and the immune response to vaccine antigens given with the adjuvant is enhanced.

### CROSS-REFERENCE TO RELATED APPLICATION

This application claims priority based upon copending U.S. Provisional Patent Application, of Ljunberg, Lladser, and Kiessling, Ser. No. 61/212,554, Filing Date 13 Apr. 2009, for USE OF VECTORS EXPRESSING INTRACELLULAR POLYNUCLEOTIDE BINDING PROTEINS AS ADJUVANTS.

### BACKGROUND OF THE INVENTION

1. Technical Field

The present invention relates generally to vaccine compositions and methods, and, more particularly, to vaccine compositions including a vaccine antigen and an activator.

2. Background Art

Genetic vaccination induces an immune response capable of protection against viral challenges and tumor rejection in small animal models alongside development of cytotoxic T lymphocytes (CTL) and/or antibodies [Holmgren et al 2006, Lindencrona et al 2004, Robinson et al 1993, Ulmer et al 1993, Vertuani et al 2004]. However, DNA vaccines have thus far failed to elicit robust immune responses in clinical trials.

The development of active immunotherapy of cancer has additionally been hampered by difficulties of breaking tolerance against weak, tumor-associated self-antigens. Therefore, efficient delivery and adjuvants are required for successful DNA immunization in humans.

DNA vaccination to the skin is an attractive approach for clinical applications as skin is easily accessible and has a high number of antigen presenting cells, e.g. Langerhans cells. In vivo intradermal DNA electroporation allows efficient plasmid uptake promoting high antigen expression and induces higher levels of specific T cells thus increasing the efficacy of DNA vaccination [Roos et al 2006]. Roos described the use of electroporation to enhance the immune response to cancer vaccines. Roos also described a specific pulse protocol called PulseAgile® that enhanced cellular immunity to cancer vaccine antigens. It would be desirable to have an adjuvant that would further boost the immune response.

Several recombinant and plasmid-encoded cytokines such as IL-2, GM-CSF, IL-12 and IL-15 have been employed as molecular adjuvants [Barouch et al 2000, Rollman et al 2004, Sin et al 1999]. These cytokines may act by increasing the inflammatory response in the vaccinated site, thus recruiting immune cells to the site of vaccination and promoting maturation of professional antigen presenting cells such as dendritic cells (DC). Single cytokines may have an immunomodulatory effect; IL-12 is a strong promoter of a Th1 type response, whereas IL-15 contributes to the induction of memory CD8⁺ T cells [Kutzler et al 2005].

Recombinant cytokines in the form of protein have a finite in vivo lifespan. Because of this, the cytokines are not concurrently present during a significant amount of time during which antigen is expressed in cells. It would be desirable to have a molecular adjuvant that is expressed concurrently with the cytokine so that its immune enhancing effect could be present a significant amount of time during which antigen is present.

Plasmid encoded cytokines delivered in a DNA vaccine mix with plasmid encoded antigen have the potential for concurrent expression. Since such plasmid encoded adjuvants would be diluted in the mixture with plasmid encoded antigen, the dose is reduced and the potential expression of the adjuvant would be reduced. Alternatively, overproduction of cytokines is possible since strong constitutive promoters are used to express cytokines from plasmids. A means to'induce production of cytokines while simultaneously modulating the maximum response would be desirable.

Cytokines act in concert, and it would be desirable to target the signaling pathways upstream of the onset of transcription of single cytokine genes. This could be achieved by activating Pattern Recognition Receptors (PRR), a diverse group of evolutionary conserved receptors designed to detect common structures shared by various pathogens, such as the Toll-Like Receptors (TLR). The bacterial TLR5 ligand flagellin from Salmonella typhimurium has been used to potentiate genetic vaccines and conferred protection to lethal Influenza challenge in vaccinated mice [Applequist et al 2005]. In addition, bacterial hypomethylated CpG-containing DNA that is recognized by TLR9 has been used in many studies including clinical trials with promising results [Krieg et al 1995, Davis et al 1998, Hemmi et al 2000, Cooper et al 2004]. Each of these Toll like receptors are receptors that exist on the surface of cells of some cells such as myeloid lineage cells. It would be desirable to utilize a receptor that can bind to cytoplasmic DNA such as that delivered into cells for DNA vaccination.

U.S. Pat. Nos. 6,239,116 and 6,406,705 describe the use of oligonucleotides containing unmethylated CpG dinucleotides (TLR9 binding ligands) as adjuvants alone or adjuvants with other non-nucleic acid adjuvants respectively. The use of vectors expressing intracellular receptors for polynucleotides as an adjuvant was not described.

The use of TLR ligands as molecular adjuvants substantiates the possibility of using ligands for other PRR as molecular adjuvants. It has been postulated that there is a PRR that would recognize intracellular DNA. Interestingly, after transfection with plasmid DNA, muscle cells upregulate expression of type I interferon, and upregulate expression of MHC class I and co-stimulatory molecules. Moreover, they can activate antigen-specific CD8+ T cells in a TLR9-independent but interferon response factor 3 (IRF3) dependent manner [Shirota et al 2007].

However molecular adjuvants such as the aforementioned ligands of Toll like receptors are delivered in the extracellular environment where they bind to Toll like receptors on the external cell membrane. In the extracellular environment the ligands to Toll like receptors degrade and thus are not present concurrently with antigen expression. It would be desirable to have the adjuvant present concurrently with the antigen.

A recently discovered, unction of the Z-DNA binding protein 1 (ZBP1 also known as DLM-1) reveals that it acts as a cytosolic PRR for double stranded DNA, and has tentatively been termed DNA-dependent activator of interferon regulatory factors (DAI) [Takaoka et al 2007]. DNA stimulation of DAI leads to type I IFN production, IL-6 upregulation and secretion of chemokine C-X-C motif ligand 10 (CXCL10). Two biochemical pathways are involved, one mediated via IRF3 activation and one via NF-κB activation [Kaiser et al. 2008]. The Takaoka publication was the first to use of the name DAI. They disclosed that DAI can bind to type B DNA (normal DNA) as well as Z-DNA. Since B-DNA is the normal DNA configuration, this would mean that DAI can bind to normal double stranded DNA. After binding of DAI to B DNA, the complex interacts, in one pathway, with IRF3 and TBK1. This in turn leads to type 1 interferon gene induction by a complex that includes IRF3 but not DAI. Thus Takaoka reveals that binding of B-DNA to DAI initiates intracellular pathways that result in coordinated secretion of selected cytokines. This process is a function of native DAI and not due to the presence of DAI beyond normal physiological levels. The use of intracellular expression vectors to increase expression of DAI beyond normal physiological levels to augment immune responses to vaccines was not discussed.

DAI expression has'been detected in many tissues; including heart, spleen, lung, liver, kidney and testis [Fu et al 1999] thus emphasizing its relevance as a sentinel for infectious diseases. DAI is a model for DNA dependent activation of IFN production. The three DNA-binding domains of DAI directly interact with cytosolic DNA, resulting in multimerization of DAI. This in turn recruits TBK1 (Tank binding Kinase 1) and IRF3 to the DNA-DAI complex. At this point, DAI is phosphorylated by TBK1, which amplifies the recruitment of TBK1 and IRF3. IRF3 is subsequently activated by phosphorylation and dimerized IRF3 becomes part of a complex that promotes type I interferon transcription and release [Wang et al 2008]. Kaiser et al. have reported that NF-κB activation occurs on polynucleotide-binding by DAI through phosphorylation of RIP-1 and RIP-3, that in turn phosphorylate the IκB inhibitor thus activating NF-κB, promoting expression of proinflammatory cytokines such as IL-6 and TNF-α and CXCL10.

DAI may not be the only cytoplasmic receptor that binds to DNA (reviewed in Takaoka 2008). At least two lines of evidence support this possibility. First, suppression of DAI expression by siRNA results in incomplete suppression of induction of type I interferon (Takaoka 2007). Second, the innate immune system in DAI knockout mice is normal indicating that there are other means to induce type I interferons.

TBK1 described in the pathway above is essential for innate and adaptive immune responses to DNA vaccines (Ishii 2008). Ishii revealed that normal immune responses to DNA vaccines were dependent on the presence of TBK1 and this dependence occurred in the absence of TLR9 receptor binding and in the absence of DAI signalling.

An additional means to activate the TBK1 pathway is a PRR pathway initiated by binding of double stranded RNA to RIG-1 (Yoneyama 2008). This pathway also leads to IRF-3 phosphorylation and dimerization and ultimately to genomic expression of type I interferons. PCT patent application number WO200808080091 describes the use of ligands that bind to RIG-1 as adjuvants. The patent application did not describe expression of RIG-1 itself in amounts above amounts existing in the cytoplasm at the time of adjuvant delivery.

U.S. Pat. No. 7,001,718 Describes the use of Z-DNA binding proteins to reduce the pathogenicity of infectious diseases. Disclosed in that patent is a method for detecting inhibitors of Z-DNA binding proteins. Also disclosed is a method for detecting/discovering anti-infective agents using Z-DNA binding proteins. The patent does not describe the use of DAI (a Z-DNA binding protein) or other DNA or RNA binding proteins as a vaccine adjuvant that can boost an immune response to a vaccine.

### OTHER PUBLICATIONS

Applequist, S. E., et al., Activation of innate immunity, inflammation, and potentiation of DNA vaccination through mammalian expression of the TLR5 agonist flagellin. J Immunol, 2005. 175(6): p. 3882-91.
Barouch, D. H., et al., Control of viremia and prevention of clinical AIDS in rhesus monkeys by cytokine-augmented DNA vaccination. Science, 2000. 290(5491): p. 486-92.
Cooper, C. L., et al., CPG 7909, an immunostimulatory TLR9 agonist oligodeoxynucleotide, as adjuvant to Engerix-B HBV vaccine in healthy adults: a double-blind phase I/II study. J Clin Immunol, 2004. 24(6): p. 693-701.
Davis, H. L., et al., CpG DNA is a potent enhancer of specific immunity in mice immunized with recombinant hepatitis B surface antigen. J Immunol, 1998. 160(2): p. 870-6.
Fu, Y., et al., Cloning of DLM-1, a novel gene that is up-regulated in activated macrophages, using RNA differential display. Gene, 1999. 240(1): p. 157-63.
Hemmi, H., et al., A Toll-like receptor recognizes bacterial DNA. Nature, 2000. 408(6813): p. 740-5.
Holmgren, L., et al., A DNA vaccine targeting angiomotin inhibits angiogenesis and suppresses tumor growth. Proc Natl Acad Sci USA, 2006. 103(24): p. 9208-13.
Ishii, K J. TANK-binding kinase-1 delineates innate and adaptive immune responses to DNA vaccines. 2008. Nature. 457: p. 725-9.
Kaiser, W. J. et al., Receptor-interacting protein homotypic interaction motif-dependent control of NF-κB activation via the DNA-dependent activator of IFN regulatory factors. 2008. J. Immunol. 181: p. 6427-34.
Kim; Y. G., et al., A role for Z-DNA binding in vaccinia virus pathogenesis. Proc Natl Acad Sci USA, 2003. 100(12): p. 6974-9.
Kutzler, M. A., et al., Communization with an optimized IL-15 plasmid results in enhanced function and longevity of CD8 T cells that are partially independent of CD4 T cell help. J Immunol, 2005. 175(1): p. 112-23.
Krieg, A. M., et al., CpG motifs in bacterial DNA trigger direct B-cell activation. Nature, 1995. 374(6522): p. 546-9.
Lindencrona, J. A., et al., CD4+ T cell-mediated HER-2/neu-specific tumor rejection in the absence of B cells. Int J Cancer, 2004. 109(2): p. 259-64.
Roos, A. K., et al., Enhancement of cellular immune response to a prostate cancer DNA vaccine by intradermal electroporation. Mol Ther, 2006. 13(2): p. 320-7.
Rollman, E., et al., Multi-subtype gp160 DNA immunization induces broadly neutralizing anti-HIV antibodies. Gene Ther, 2004. 11(14): p. 1146-54.
Robinson, H. L., L. A. Hunt, and R. G. Webster, Protection against a lethal influenza virus challenge by immunization with a haemagglutinin-expressing plasmid DNA. Vaccine, 1993. 11(9): p. 957-60.
Shirota, H., et al., Potential of transfected muscle cells to contribute to DNA vaccine immunogenicity. J Immunol, 2007. 179(1): p. 329-36.
Sin, J. I., et al., In vivo: modulation of vaccine-induced immune responses toward a Th1 phenotype increases potency and vaccine effectiveness in a herpes simplex virus type 2 mouse model. J Virol, 1999. 73(1): p. 501-9.
Smith, E. J., et al., IRF3 and IRF7 phosphorylation in virus-infected cells does not require double-stranded RNA-dependent protein kinase R or lkappa B kinase but is blocked by Vaccinia virus E3L protein. J Biol Chem, 2001. 276(12): p. 8951-7.
Takaoka, A., et al., DAI (DLM-1/ZBP1) is a cytosolic DNA sensor and an activator of innate immune response. Nature, 2007. 448(7152): p. 501-5.
Takaoka A, et al., Cytosolic DNA recognition for triggering innate immune responses. Adv. Drug Del. Rev., 2008; 60:847-857
Ulmer, J. B., et al., Heterologous protection against influenza by injection of DNA encoding a viral protein. Science, 1993. 259(5102): p. 1745-9.
Vertuani, S., et al., Improved immunogenicity of an immunodominant epitope of the HER-2/neu protooncogene by alterations of MHC contact residues. J Immunol, 2004. 172(6): p. 3501-8.
Wang, Z., et al., Regulation of innate immune responses by DAI (DLM-1/ZBP1) and other DNA-sensing molecules. Proc Natl Acad Sci USA, 2008. 105(14): p. 5477-82.
Xiang, Y., et al., Blockade of interferon induction and action by the E3L double-stranded RNA binding proteins of vaccinia virus. J Virol, 2002. 76(10): p. 5251-9.
Yoneyama M, Onomoto K, Fujita T. Cytoplasmic recognition of RNA. Adv. Drug Del. Rev. 2008. 60:841-846

### DEFINITIONS

Adjuvant: Any substance that enhances an immune response to a vaccine.

Antigen: Immunogen or a substance that induce a response from the immune system.

B-DNA: Normal DNA with a right hand helical twist and a major and minor groove.

CpG: A sequence in DNA where a cytosine is followed by a guanidine. The p represents the phosphate binding the two molecules. The CpG is often methylated in eukarylotic cells and often un-methylated in prokaryotic cells.

CTL: cytotoxic T lymphocyte

DAI: DNA dependent activator of interferon regulatory factors. It also is known as Z-DNA binding protein (ZBP1) and DLM-1. In addition to binding Z-DNA, DAI also is known to bind to B-DNA.

pDAI: a plasmid capable of expressing DAI.

DNA: Deoxyribonucleic acid

Expression vector: For this invention, an expression vector is any polynucleotide whose presence in cells results in the production of a desired protein. This includes plasmids with expression sequences controlled by promoters and enhancers. It also includes promoter free sequences such as messenger RNA whose presence in cells results in production of a desired protein.

IFN-α/βR−/−: Mice that are interferon alpha receptor and interferon beta receptor negative on both alleles

IFN: Interferon

IL: interleukin

Immunomodulators Any protein that influences the immune system. Cytokines and chemokines are immunomodulators.

IRF3: Interferon regulatory factor 3

IRF3−/− mice: Mice lacking expression of interferon regulatory factor 3 on both alleles.

Mda-5 or MDA5: Melanoma differentiation-associated antigen 5.

Pharmaceutically acceptable carrier: Any substance in which a drug or vaccine can be suspended for injection. The substance can be in a solid state, liquid state or a mixture of solids and liquids.

Polynucleotide: Any polymer of RNA or DNA. It can be either single stranded or double stranded.

PRR: pattern recognition receptors

Pulse Agile®: A sequence of at least three waveforms that has one, two, or three of the following characteristics (1) at least two of the at least three waveforms differ from each other in waveform amplitude, (2) at least two of the at least three waveforms differ from each other in waveform width, and (3) a first waveform interval for a first set of two of the at least three waveforms is different from a second waveform interval for a second set of two of the at least three waveforms.

RIG-1: Retinoic acid inducible gene 1 protein

TBK1: tank binding kinase. It is one of the molecules in the downstream pathway of DAI leading to activation of regulators of type 1 interferon expression.

TLR: toll like receptor. A family of pattern recognition receptors characterized by an extracellular leucine-rich domain and a cytoplasmic domain that share homology with the interleukin 1 receptor and the drosophila toll protein. Following pathogen recognition, toll-like receptors recruit and activate a variety of signal transducing adaptor proteins (http://www.reference.md/files/D051/mD051193.html). TLR9 is a receptor whose ligand is un-methylated CpG.

Z-DNA: A transiently appearing form of DNA where the helical twist is left and there is little difference between the major and minor grooves.

### SUMMARY OF THE INVENTION

For the purposes of this invention overexpressing means expressing an amount that is in addition to existing cellular levels.

The present invention relates to methods and products for enhancing an immune response using a novel molecular adjuvant. The invention is useful in one aspect as a method of enhancing an antigen specific immune response in a subject. The method includes the steps of administering to the subject a combination of adjuvant and vaccine antigen in order to induce an immune response in the subject. The method includes transfecting cells in vivo with an RNA or DNA expression vector capable of expressing a protein that has a polynucleotide sensing capability and can activate an intracellular pathway ultimately leading to activation of genomic transcription of cytokines to include type 1 interferons. Expression of the polynucleotide binding protein is in addition to normal cellular levels of this protein. This novel process results in cytokine expression that is concurrent with antigen expression and is broader than expression of a single cytokine.

Important classes of intracellular proteins capable of up-regulating cellular cytokine secretion are those that bind to polynucleotides in the cytoplasm. Some proteins with this function bind to DNA and some bind to RNA. One purpose for these proteins is to detect viral polynucleotides in the cytoplasm and to alert the cell to the presence of the virus. Binding of the polynucleotide to its cytosolic sensor initiates a biochemical pathway that leads to type I interferon production in an IRF3 dependent fashion, and IL-6 upregulation likely in an NF-κB dependent fashion.

In this respect, in accordance with the invention, these proteins are ideal candidates as adjuvants when overexpressed in cells. In addition, polynucleotide vectors expressing vaccine antigen and polynucleotide vectors expressing vaccine adjuvant remaining in the cytoplasm bind to the overexpressed polynucleotide binding protein and thus become part of the complex that initiates intracellular pathways that induce expression of cytokines.

An adjuvant of the invention is a polynucleotide vector capable of expressing a DNA dependent activator of type 1 interferon. One DNA dependent activator of interferon is known by three different names, ZBP-1 (Z DNA binding protein one), DLM-1, and DAI (DNA-dependent activator of interferon regulatory factors). The sequence of the protein and the gene that encodes DAI is found at the National Library of Medicine database with reference number BC131706 or GenBank ID NM_{—}030776. In accordance with the invention, changes to the polynucleotide sequence that do not modify the functional activity of the expressed protein may be made without changing the use of the expressed protein as an adjuvant.

In accordance with the invention, the DAI adjuvant may be administered with a DNA vaccine antigen in the form of an expression vector capable of expressing DAI. One or more DNA vaccine antigen expression vectors capable of expressing one or more vaccine antigens may be mixed with an expression vector capable of expressing DAI. In accordance with the invention, the mixture of expression vectors containing vectors capable of expressing both antigens and DAI may be injected into living tissue for the purpose of expressing all of these proteins in vivo.

Another adjuvant of the invention is a polynucleotide vector capable of expressing an RNA dependent activator of interferon. Examples of RNA dependent activators of interferon are RIG-1 and Mda-5. Both of these proteins are members of a group of proteins called CARD (caspase recruitment domain) proteins. A representative RIG-1 protein and nucleotide sequence is found at GenBank with reference number AF092922.1. A representative Mda-5 protein and nucleotide sequence is found at GenBank with reference number AF095844.1.

In accordance with the invention, the RIG-1 and Mda-5 adjuvants may be administered with an RNA vaccine antigen in the form of an expression vector capable of overexpressing RIG-1 and Mda-5. One or more DNA or RNA vaccine antigen expression vectors capable of expressing one or more vaccine antigens may be mixed with an expression vector capable of overexpressing RIG-1 and Mda-5. The mixture of expression vectors containing vectors capable of expressing both antigens and the cytosolic RNA binding protein may be delivered to cells in living tissue for the purpose of expressing all of these proteins in vivo.

### BRIEF DESCRIPTION OF THE DRAWINGS

FIG. 1 describes Prior Art mechanisms by which DAI activates innate immunity. DAI is a cytosolic DNA sensor that has been described to induce production of type I interferons (IFNs) and other proinflammatory cytokines via two distinct signalling pathways. Binding of dsDNA to DAI induces the recruitment and phosphorylation of TBK1 with subsequent/sequential activation of the transcription factor IRF3 that leads to expression of IFN-α and IFN-β target genes. The other pathway involves the association of DAI with RIP-1 (and also RIP3) and phosphorylation of IKB leading to the activation of the transcription factor NFκB and transcription of type I IFNs as well as pro-inflammatory genes such as IL-6 and TNF-α.

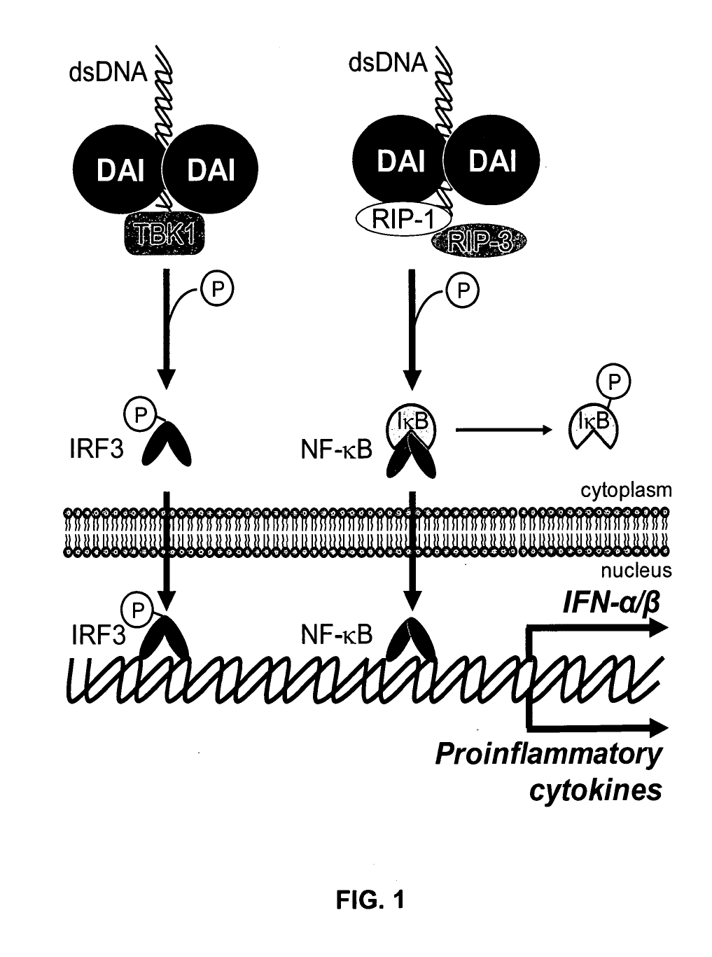

FIG. 2 is a plasmid map of pDAI showing insertion of a DAI gene into a pVAX expression vector.

FIG. 3 shows rapid up-regulation of type I IFN mRNA levels in skin of mice injected with pDAI adjuvant delivered by electroporation.

FIG. 4 shows up-regulation of proinflammatory mRNA levels in skin of mice injected with pDAI adjuvant delivered by electroporation.

FIG. 5 shows that mice co-immunized with pDAI and a TRP2 encoding vector (pTRP2) have a stronger CTL response than mice immunized with the pTRP2 vector alone.

FIG. 6 shows that CD8+ T cells in mice immunized with the pDAI and pTRP2 up-regulate degranulation marker CD107 on the cell surface and thus display/have a cytotoxic phenotype.

FIG. 7 shows that the enhancement of CD8+ T cell response in IRF3−/− and IFN-α/βR−/− mice co-immunized with pDAI and pTRP2 is dependent on interferon response factor 3 (IRF3) and type I IFN effects.

FIG. 8 shows that pDAI co-immunization increases antibody responses in mice to TRP2. Antibodies were bound to cells transfected with the TRP2 gene and detected with an anti-mouse anti-IgG FITC-conjugated antibody by flow cytometry analysis.

FIG. 9 shows that mice co-immunized with pDAI and pTRP2 have improved survival to challenge with B16 tumor cells compared to pTRP2 only or control animals.

FIG. 10 shows improved survival of mice co-immunized with pDAI and pTRP2 compared to pTRP2 is dependent of IRF3.

FIG. 11 shows that pDAI+pTRP2 immunization induces long-term protection in mice to re-challenge.

FIG. 12 shows that emergence of autoimmune vitiligo is faster after immunization with pTRP2 when mice are co-immunized with pDAI.

FIG. 13 shows that mice co-immunized with pDAI and survivin encoding plasmid (pSURV) have a stronger T cell response than mice immunized with pSURV alone.

FIG. 14 shows that the adjuvant effect of pDAI to pSURV in mice is not mediated by IRF3, but is mediated by type I interferons.

FIG. 15 shows that mice co-immunized with pDAI and pSURV have improved survival to challenge with B16 tumor cells compared to pSURV only or control animals.

### DETAILED DESCRIPTION OF THE INVENTION

Some vaccine antigens are poorly immunogenic. An example of a class of such antigens is tumor antigens where the antigen may be very similar to native proteins. Simultaneous'expression of adjuvants of this invention with vaccine antigens induces expression of cytokines that include type 1 interferons. The cytokines in turn boost the antigen specific immune response induced by the presence of vaccine antigens.

The present invention relates to methods and products for enhancing an immune response using a novel molecular adjuvant. The invention is useful in one aspect as a method of enhancing an antigen specific immune response in a subject. The method includes the steps of administering to the subject a combination of adjuvant and vaccine antigen in order to induce an immune response in the subject. The method includes transfecting cells in vivo with an RNA or DNA expression vector capable of expressing a protein that has a polynucleotide (ssDNA, dsDNA, ssRNA, or dsRNA) sensing capability and can activate an intracellular pathway ultimately leading to activation of genomic transcription of cytokines to include type 1 interferons. Other immunomodulators may also be expressed as direct or indirect byproducts of this pathway. Expression of the polynucleotide binding protein is in addition to normal cellular levels of this protein. This novel process results in cytokine expression that is significantly concurrent with antigen expression and is broader than expression of a single cytokine.

An adjuvant of the invention is a polynucleotide vector capable of expressing a DNA dependent activator of type 1 interferon. One DNA dependent activator of interferon is known by three different names, ZBP-1 (Z DNA binding protein one), DLM-1, and DAI (DNA-dependent activator of interferon regulatory factors). The sequence of the protein and the gene that encodes DAI is found at the National Library of Medicine database with reference number BC131706 or GenBank ID NM_{—}030776. It is recognized that changes to the polynucleotide sequence that do not modify the functional activity of the expressed protein may be made without changing the use of the expressed protein as an adjuvant.

The DAI adjuvant may be administered with a DNA vaccine antigen in the form of an expression vector capable of expressing DAI. One or more DNA vaccine antigen expression vectors capable of expressing one or more vaccine antigens may be mixed with an expression vector capable of expressing DAI. The mixture of expression vectors containing vectors capable of expressing both antigens and DAI may be injected into living tissue for the purpose of expressing all of these proteins in vivo.

Another adjuvant of the invention is a polynucleotide vector capable of expressing an RNA dependent activator of interferon. Examples of RNA dependent activators of interferon are RIGA and Mda-5. Both of these proteins are members of a group of proteins called CARD (caspase recruitment domain) proteins. A representative RIGA protein and nucleotide sequence is found at GenBank with reference number AF092922.1. A representative Mda-5 protein and nucleotide sequence is found at GenBank with reference number AF095844.1.

The RIGA and Mda-5 adjuvants may be administered with an RNA vaccine antigen in the form of an expression vector capable of overexpressing RIGA and Mda-5. One or more DNA or RNA vaccine antigen expression vectors capable of expressing one or more vaccine antigens may be mixed with an expression vector capable of overexpressing RIG-1 and Mda-5. The mixture of expression vectors containing vectors capable of expressing both antigens and the cytosolic RNA binding protein may be delivered to cells in living tissue for the purpose of expressing all of these proteins in vivo.

A mixture of expression vectors capable of expressing vaccine antigen and adjuvant can be delivered in a number of ways. One method is simply injection of the mixture into tissues where some of the expression vectors enter cells. However this process is not very efficient and other means may be employed to increase the efficiency. Examples are electroporation, biolistic delivery and sonoporation. Chemical means include cationic lipids, cationic polymers, nanoparticles and others. There are other delivery enhancement methodologies that may be used as the examples are not limiting.

Expressed antigens used with adjuvants of this invention may be of any type known in the art. For example, the antigen may be selected from the group consisting of peptides, polypeptides, glycoproteins, transmembrane proteins and any other protein that can be expressed in mammalian cells.

Another embodiment of the invention is separate administration of the adjuvant. For example the adjuvant may be administered one or more days prior to or after administration of the specific vaccine antigen at identical or close sites.

Another embodiment of the invention is the administration of an expression vector capable of expressing adjuvants of this invention with antigens other than antigens expressed by expression vectors. Antigens selected for this embodiment may be selected from the group consisting of polypeptides, proteins, polysaccharides, haptens, glycosylated proteins, lipoproteins, lipopolysaccharides, cells, cell extracts, polysaccharide conjugates, lipids, glycolipids and carbohydrates. Such antigens may be administered in various forms of purification from crude to highly purified. The antigens may be isolated from natural products, fermentation products or a product of recombinant DNA technology expressed in various types of cells. Antigens may also be synthetic peptides.

The invention may be used to affect the quality as well as the quantity of the immune response. For instance, an effective immune response to many infectious diseases, to include viral and parasitic, is often of Th-1 type. The vector capable of expressing adjuvants of this invention may be used to promote a Th-1 type immune response.

The term expression vector may include but is not limited to plasmid DNA with an insert encoding antigen plus essential elements such as promoters, promoter enhancers and polyadenylation sites. The term expression vector may also include viral vectors, including DNA or RNA type viruses. The term expression vector also includes messenger RNA.

The inclusion of the aforementioned adjuvant of this invention with vaccine antigen in a vaccine does not limit inclusion of additional components such as excipients, pharmaceutically acceptable carriers, other adjuvants or other expression modulators such as siRNA, is RNA or oligonucleotides. Pharmaceutically acceptable carriers can be any material suitable for use with injectable vaccines, drugs or medications. Non-limiting examples of pharmaceutically acceptable carriers include water, PBS, polyethylene polymers, polyvinyl chloride gels, saccharides, polysaccharides and many other substances.

Another aspect of the invention is a vaccine composition, comprising a vaccine antigen, and a polynucleotide capable of expressing amounts of an activator of interferon regulatory factors, wherein said amounts of said activator are in addition to existing cellular levels of the activator.

This invention in one aspect is a discovery that overexpression of a DNA dependent regulator of interferon expression enhances immune responses to an antigen.

A preferred embodiment of the invention is the use of a plasmid capable of expressing DAI as an adjuvant. The plasmid vector capable of expressing DAI can be prepared using standard molecular biological techniques such and those described in Molecular Cloning: A Laboratory Manual, 3^{rd} Edition (CSHL Press) which is incorporated in its entirety by reference. The DAI expressing vector is mixed with vectors capable of expressing desired antigens in mammalian cells. Examples of antigens can be selected from a list that includes infectious disease antigens, cancer antigens, self antigens or any other protein antigen. Plasmids are mixed with a pharmaceutically acceptable carrier and stored in a liquid or dried state. A preferred means of delivery of the mixture of plasmids capable of expressing DAI adjuvant and vaccine antigens is electroporation. For electroporation delivered plasmids, the plasmid mixture can either be injected first intradermally into skin or simultaneously inserted with electroporation electrodes. After injection, if not already in the tissue, electroporation electrodes are inserted spanning the injected area. After insertion of electroporation electrodes, pulsed electric fields are applied. A preferred electric field is a Pulse Agile® electric field. An example of a specific effective Pulse Agile® electric field is two pulses of 1125 V/cm, 50 pS duration, (200 pS pulse interval for the first pulse and 50 mS interval for the second pulse) followed by eight pulses of 275 V/cm, 10 mS duration and 20 mS pulse intervals. An electroporation system capable of delivering the described pulses μs Derma Vax™ made by Cyto Pulse Sciences Inc., Glen Burnie, Md., USA.

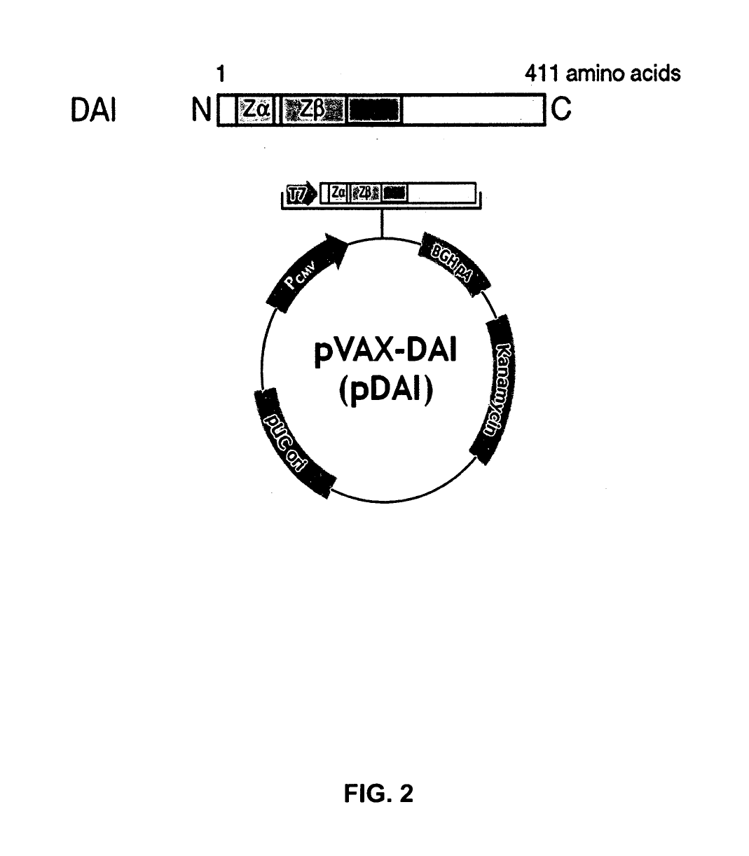

For experiments involving delivery of a plasmid capable of expressing DAI to mice, the mouse DAI gene was cloned into the pVAX vector. FIG. 2 shows a plasmid map of the gene insertion into a pVAX expression vector.

The DAI gene was cloned from isolated mouse splenocytes. Total RNA was isolated using the RNeasy kit (Qiagen). cDNA synthesis was performed with 50 ng (1 μl) of random hexamer primers, 1 μl 10 mM dNTP and 11 μl of the RNA. The RNA/primer mix was heated to 65° C. for 5 minutes. Cool on ice and then add 4 μl of first strand buffer, 1 μl DTT, 1 μl RNase out and 1 μl Superscript III reverse transcriptase. The reaction occurred at 25° C. for 5 minutes, then at 50° C. for 1 h. The enyzme was inactivated at 70° C. for 15 minutes. The cDNA was subsequently used for PCR reactions using the DAI specific primers: AGT CGA ATT CCC ACC ATG GCA GAA GCT CCT GTT GAC and AGT CGC GGC CGC TCA TTG CTT GCT CAG TCC TGT. 1.25 μl of each primer at 10 mM were mixed with 10 μl Herculase buffer (Stratagene), 0.8 μl 25 mM dNTP, 33.7 μl water, 1 μl Herculase enzyme. The PCR reaction was heated to 95° C. and run as follows: 95° C. 30 s, 55° C. 30 s, 72° C. for 90 s repeated in 35 cycles, then at 72° C. for 7 minutes. The PCR reaction was run on a 1.5% agarose gel, and the band corresponding to the size of DAI (1.2 kb) was excised and purified using the Qiagen gel extraction spin kit. DAI was subsequently cloned in to a cloning vector, pCR-Blunt (invitrogen) by mixing 4 μl of the DAI PCR product, 1 μl of the pCR-Blunt vector, 1 μl 10× ligation buffer, 3 μl of water and 1 μl of Invitrogen T4 DNA ligase. The reaction was performed at 16° C. for 1 h. 2 μl of the ligation mix was then transferred to a tube containing 50 μl of competent E. coli and kept on ice for 30 minutes before heatshock at 42° C. for 90 s. The transformation was cooled down on ice for 2 minutes before 450 μl of room temperature SOC medium was added, and the transformed bacteria were grown for 1 h at 37° C., and 100 μl of the cells were plated on pre-warmed kanamycin containing (50 μg/ml) agar plates, and grown at 37° C. over night. Next, colonies were picked from the plate and grown in LB medium at 37° C. over night before plasmid DNA was extracted (Qiagen spin miniprep kit) and clones were identified for insert by NotI and EcoRI digestion and agarose gel electrophoresis. The NotI/EcoRI digestion band corresponding to the size of DAI (1.2 kb) was excised and purified using the Qiagen gel extraction spin kit. The purified DAI DNA was then cloned into the pVAX vector that had been digested by NotI/EcoRI and purified as above. Ligation was performed with 1 μl of pVAX DNA and 4 μl of DAI DNA, 1 μl of T4 buffer, 3 μl water and 1 μl T4 DNA ligase at 16° C. for 1 h. 2 μl of the ligation mix was then transferred to a tube containing 50 μl of competent E. coli and kept on ice for 30 minutes before heat-shock at 42° C. for 90 s. The transformation was cooled down on ice for 2 minutes before 450 μl of room temperature SOC medium was added, and the transformed bacteria were grown for 1 h at 37° C., and 100 μl of the cells were plated on pre-warmed kanamycin containing (50 ug/ml) agar plates, and grown at 37° C. over night. Next, colonies were picked from the plate and grown in LB medium at 37° C. over night before plasmid DNA was extracted (Qiagen spin miniprep kit) and clones were identified for insert by NotI and XbaI digestion and agarose gel electrophoresis. Clones with the correct restriction pattern (bands at 0.3, 1.0 and 3.5 kb) were selected and sequenced with vector specific primers (T7, TAA TAC GAC TCA CTA TAG GG; and BGH, TAG AAG GCA CAG TCG AGG).

### Example 1

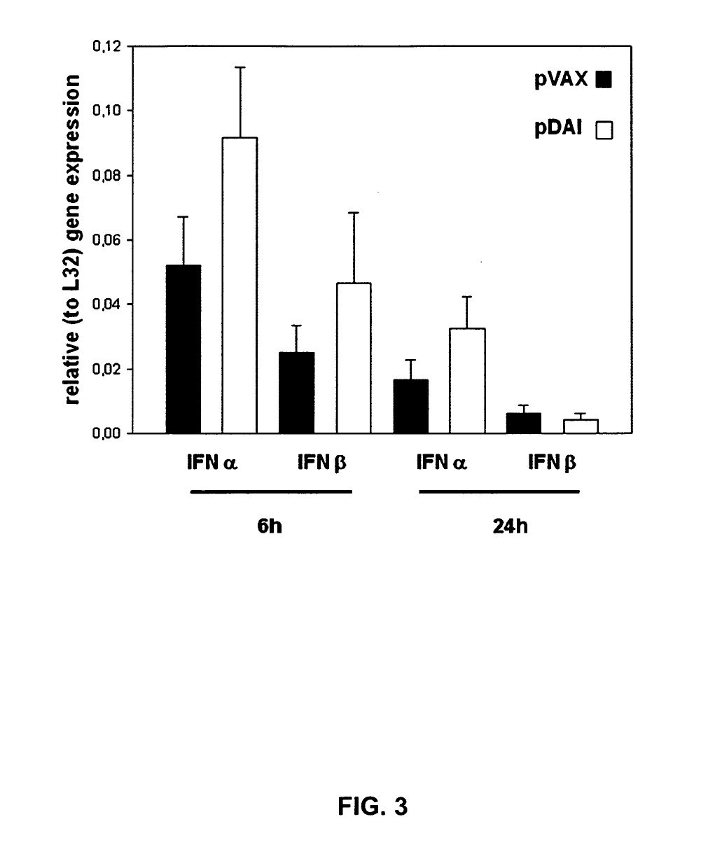

Up-regulation of type I IFN mRNA levels in skin of mice injected with pDAI adjuvant delivered by electroporation (FIG. 3)

C57BL/6 mice were anesthetized with isoflurane and immunized once with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pUlses) were applied using Derma Vax™ Intradermal Delivery System (Cyto Pulse Sciences, Inc). Mice were injected with 40 μg empty vector (pVAX, n=6), or 20 μg DAI encoding vector plus 20 μg of empty vector (pDAI, n=6). Either 6 or 24 hours after DNA electroporation, skin samples were taken from injection sites and stored in 2 ml of RNAlater RNA Stabilization Reagent (Qiagen) at 4° C. Total RNA was isolated from murine skin using RNeasy Mini Kit (Qiagen) and cDNA prepared (iScript cDNA synthesis kit; Bio-Rad; Hercules, Calif.) according to the manufactor's instructions. Messenger RNA levels were quantified by real-time quantitative PCR (iQ SYBR Green Supermix; Bio-Rad, AB17500; Applied Biosystems, Carlsbad, Calif.) using a two-step cycling program (1 min at 95° C., followed by 40 cycles of 15 sec at 95° C. and 1 min at 62-64° C.). Relative gene expression for IFN-α and IFN-β was determined by normalizing the expression of each target to the L32 housekeeping gene. (*P<0.05, Student's t-test). Error bars represent the standard error of the mean.

| Primers | Sequences |
|---|---|
| IFN-α forward | 5′-ATGGCTAGRCTCTGTGCTTTCCT-3′ |
| IFN-α reverse | 5′-AGGGCTCTCCAGAYTTCTGCTCTG-3′ |
| IFN-β forward | 5′-CATCAACTATAAGCAGCTCCA-3′ |
| IFN-β reverse | 5′-TTCAAGTGGAGAGCAGTTGAG-3′ |
| IFN-γ forward | 5′-GCTTTAACAGCAGGCCAGAC-3′ |
| IFN-γ reverse | 5′-GCAAGCACCAGGTGTCAAGT-3′ |

The results are shown in FIG. 3. Type 1 interferons were significantly upregulated by transfection of mouse skin with a plasmid capable of expressing DAL Six hours after injection, increased levels of IFAα and IFNβ were seen.

### Example 2

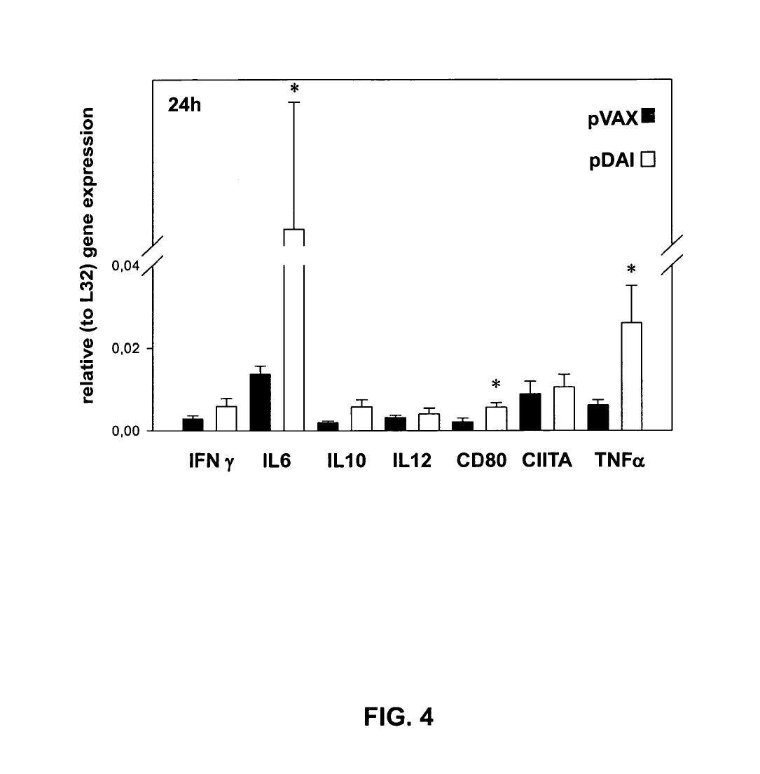

Up-regulation of proinflammatory mRNA levels in skin of mice injected with pDAI adjuvant delivered by electroporation (FIG. 4)

C57BU6 mice were anesthetized with isoflurane and immunized once with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs and electric pulses (two 1125 V/cm pulses followed by eight 275 V/cm pulses) were applied using Derma Vax™ Intradermal Delivery System (Cyto Pulse Sciences, Inc). Mice were injected with 40 μg empty vector (pVAX, n=6), or 20 μg DAI encoding vector plus 20 μg of empty vector (pDAI, n=6). 24 hours after DNA electroporation, skin samples were taken from injection sites and stored in 2 ml of RNAlater RNA Stabilization Reagent (Qiagen) at 4° C. Total RNA was isolated from murine skin using RNeasy Mini Kit (Qiagen) and cDNA prepared (iScript cDNA synthesis kit; Bio-Rad; Hercules, Calif.) according to the manufactors instructions. Messenger RNA levels were quantified by real-time quantitative PCR (iQ SYBR Green Supermix; Bio-Rad, ABI7500; Applied Biosystems, Carlsbad, Calif.) using a two-step cycling program (1 min at 95° C., followed by 40 cycles of 15 sec at 95° C. and 1 min at 62-64° C.). Relative gene expression for CIITA, CD80, IFN-γ, IL6, IL10, 102, TNF-α was determined by normalizing the expression of each target to the L32 housekeeping gene. (*P<0.05, Student's t-test). Error bars represent the standard error of the mean.

| Primers | Sequences |
|---|---|
| CD80 forward | 5′-CCCCAGAAGACCCTCCTGATAG-3′ |
| CD80 reverse | 5′-CCGAAGGTAAGGCTGTTGTTTG-3′ |
| CIITA forward | 5′-TGCAGGCGACCAGGAGAGACA-3′ |
| CIITA reverse | 5′-GAAGCTGGGCACCTCAAAGAT-3′ |
| IFN-γ forward | 5′-GCTTTAACAGCAGGCCAGAC-3′ |
| IFN-γ reverse | 5′-GCAAGCACCAGGTGTCAAGT-3′ |
| IL-6 forward | 5′-GATGCTACCAAACTGGATATAATC-3′ |
| IL-6 reverse | 5′- |
| | GGTCCTTAGCCACTCCTTCTGTG-3′ |
| IL-10 forward | 5′- |
| | GTGAAAATAAGAGCAAGGCAGTG-3′ |
| IL-10 reverse | 5′ - |
| | ATTCATGGTCTTGTAGACACC-3′ |
| IL-12 forward | 5′-AGTACCCTGTGCCTTGGTAG- |
| | 3′ |
| IL-12 reverse | 5′-GATTCTGAACTGCGTTG-3′ |
| TNF-α forward | 5′- |
| | CTACAGGCTTGTCACTCGAATT-3′ |
| TNF-α reverse | 5′- |
| | AATGGCCTCCCTCTCATCAGT-3′ |

The results are shown in FIG. 4. Increased levels of IL-6 and TNFα mRNA were noted in skin taken from the injection site 24 hours after treatment. Thus a plasmid capable of expressing DAI, delivered by electroporation into skin cells induces secondary expression of cytokines other than type 1 interferons.

### Example 3

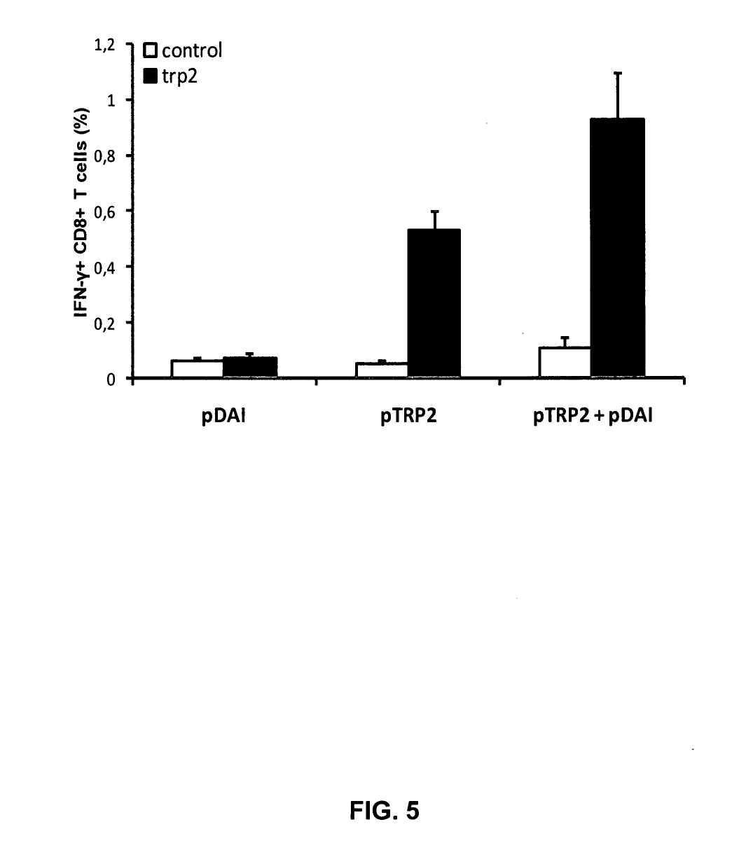

Mice co-immunized with pDAI and TRP2 encoding vector (pTRP2) have a stronger CTL response than mice immunized with the pTRP2 vector alone (FIG. 5).

C57BL/6 mice were anesthetized with isoflurane and immunized two times, 2 weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=8), 20 μg vector encoding human Tyrosine Related Protein 2 plus 20 μg empty vector (pTRP2, n=8), or 20 μg of a vector encoding DAI plus 20 μg pTRP2 (pDAI+pTRP2, n=8). 13 days after the second immunization, lymphocytes from peripheral blood were isolated as follows. Mice were bled from the tail and ˜300 μl blood was collected into tubes containing 100 μl heparin solution. Erythrocytes were lysed by adding 1 ml Ammonium Chloride (ACK) Buffer (1×Pharm Lyse BD) for 5 min at room temperature. 10-12 ml of media was added and samples were centrifuged at 400 g for 5 min. The supernatant was discarded, and the cell pellet was resuspended in 7 ml complete cell culture medium (RPMI1640, 10% FBS, Sodium pyruvate, penicillin/streptomycin and Non-essential amino acids) and centrifuged at 400 g for 5 min. The cell pellet was resuspended in 200 μl cell culture media (cell concentration should be 500,000 cells per-1000) and 100 μl were transferred per well to a U-bottom 96 well cell culture plate. 100 μl of TRP2 or control peptides at a concentration of 2.5 μM in complete medium were added per well. After 2 h of incubation at 37° C., 5% CO₂, 50 μl GolgiPlug (BD) in dilution 1:200 was added per well.

Then, the incubation proceeded for an additional 6 h at 37° C. 1 μA Fc-block (BD) in 19 μl medium was added per well 20 minutes prior to cells being harvested by centrifugation at 400 g, 5 min at 4° C. Cells were washed once with 200 μl PBS-1% FBS, and stained with 1 μl PE-conjugated anti-mouse CD8 antibody in 19 μl PBS-1% FBS. Staining proceeded for 30 min at 4° C. in the dark. Then, cells were washed twice with 200 μl PBS-1% FBS, and collected by centrifugation (400 g, 5 min, 4° C.). Cells were fixed in 100 μl of Cytofix/Cytoperm™ solution (BD) for 20 min at 4° C. in the dark, and then washed two times with 1×Perm/Wash™ solution (BD), before staining for interferon gamma (IFN-γ). 20 μl of Perm/Wash™ solution containing 1 μl anti-mouse IFN-γ FITC-conjugated (BD) antibody was added to the cells for 30 min at 4° C. in the dark. Then, cells were washed two times with 1×Perm/Wash™ solution (1 ml/wash for staining in tubes) and resuspended in PBS-1% FBS prior to flow cytometry analysis.

Results are shown in FIG. 5. In mice immunized only with pDAI, CD8+ T cells showed only background IFN-γ production upon stimulation with the TRP2 peptide, whereas mice immunized with the pTRP2 plasmid had a low but detectable response. The T cell response in mice that were co-immunized with pDAI and pTRP2, the CD8+ T cell response was significantly increased (*P<0.05, D'Agostino & Pearson omnibus normality test followed by Student's t-test). One representative of two independent experiments is shown. Error bars represent the standard error of the mean.

### Example 4

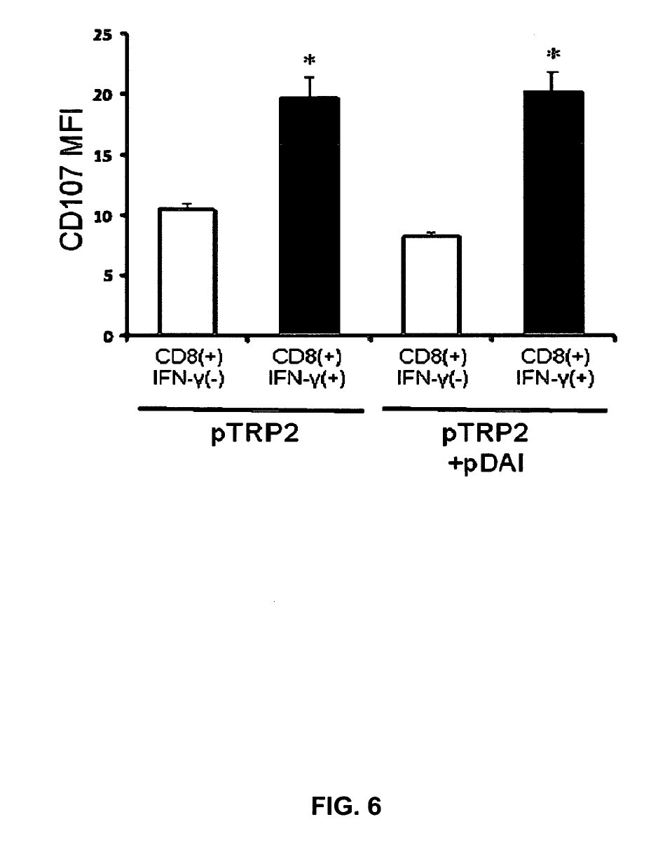

CD8+ T cells in mice immunized with pTRP2 up-regulate degranulation marker CD107 on the cell surface and thus have a cytotoxic phenotype (FIG. 6).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=9), 20 μg vector encoding human Tyrosin Related Protein 2 plus 20 μg empty vector (pTRP2, n=9), or 20 μg of a vector encoding DAI plus 20 μg pTRP2 (pDAI+pTRP2, n=9). 13 days after the second immunization, lymphocytes from peripheral blood were isolated as follows. Mice were bled from the tail and ˜300 μl blood was collected into tubes containing 100 μl heparin solution. Erythrocytes were lysed by adding 1 ml Ammonium Chloride (ACK) Buffer (1×Pharm Lyse BD) for 5 min a room temperature. 10-12 ml of media was added and samples were centrifuged at 400 g for 5 min. The supernatant was discarded, and the cell pellet was resuspended in 7 ml complete cell culture medium (RPMI1640, 10% FBS, Sodium pyruvate, penicillin/streptomycin and Non-essential amino acids) and centrifuged at 400 g for 5 min. The cell pellet was resuspended in 200 μl cell culture media (cell concentration should be 500,000 cells per 100 μl) and 100 μl were transferred per well to a U-bottom 96 well cell culture plate. 100 μl of medium containing 2.5 μM TRP2 or control peptide, and 1:100 dilution of an anti-mouse anti CD107a FITC-conjugated was added per well. After 2 h of incubation at 37° C., 5% CO₂, 50 μl GolgiPlug (BD) in dilution 1:200 was added per well. Then, the incubation proceeded for an additional 6 h at 37° C. 1 μl Fc-block (BD) in 19 μl medium was added per well 20 minutes prior to cells were harvested by centrifugation at 400 g, 5 min at 4° C. Cells were washed once with 200 μl PBS-1% FBS, and stained with 1 μl PE-conjugated anti-mouse CD8 antibody in 19 μl PBS-1% FBS. Staining proceeded for 30 min at 4° C. in the dark. Then, cells were washed twice with 200 μl PBS-1% FBS, and collected by centrifugation (400 g, 5 min, 4° C.). Cells were fixed in 100 μl of Cytofix/Cytoperm™ solution (BD) for 20 min at 4° C. in the dark, and then washed two times with 1×Perm/Wash™ solution (BD), before staining for interferon gamma (IFN-γ). 20 μl of Perm/Wash™ solution containing 1 μl anti-mouse IFN-γ APC-conjugated (BD) antibody was added to the cells for 30 min at 4° C. for in the dark. Then, cells were washed 2 times with 1×Perm/Wash™ solution (1 ml/wash for staining in tubes) and resuspended in PBS-1% FBS prior to flow cytometry analysis.

The results are shown in FIG. 6. CD8+ T cells from pTRP2 and pDAI+pTRP2 immunized mice expressed similar levels of degranulation marker CD107 as measured by mean fluorescence intensity (MFI), and did thus not display a difference in cytotoxic phenotype. (*P<0.05, Student's t-test). Error bars represent the standard error of the mean.

### Example 5

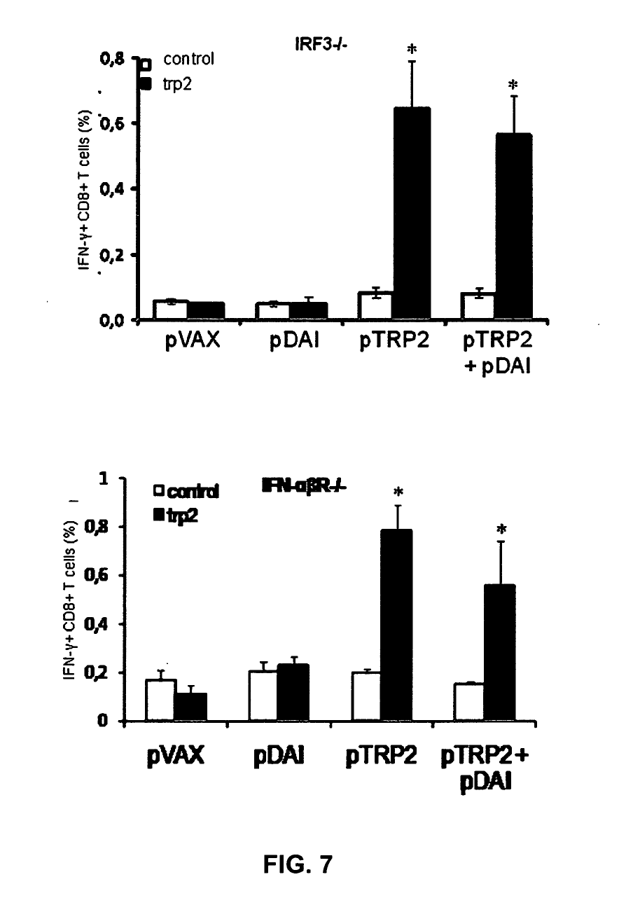

The enhancement of CD8+ T cell response in mice co-immunized with pDAI and pTRP2 is dependent on interferon response factor 3 (IRF3) and type I IFN effects (FIG. 7).

IFN-α/βR−/− (n=8 per group) and IRF3−/− (n=6 per group) mice were anesthetized with isoflurane and immunized two times, 2 weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 40 μg empty vector (pVAX), 20 μg DAI encoding vector plus 20 μg of empty vector (pDAI), 20 μg vector encoding human Tyrosin Related Protein 2 plus 20 μg empty vector (pTRP2), or 20 μg of a vector encoding DAI plus 20 μg pTRP2 (pDAI+pTRP2). 13 days after the second immunization, lymphocytes from peripheral blood were isolated as follows. Mice were bled from the tail and ˜300 μl blood was collected into tubes containing 100 μl heparin solution. Erythrocytes were lysed by adding 1 ml Ammonium Chloride (ACK) Buffer (1×Pharm Lyse BD) for 5 min a room temperature. 10-12 ml of media was added and samples were centrifuged at 400 g for 5 min. The supernatant was discarded, and the cell pellet was resuspended in 7 ml complete cell culture medium (RPMI1640, 10% FBS, Sodium pyruvate, penicillin/streptomycin and Non-essential amino acids) and centrifuged at 400 g for 5 min. The cell pellet was resuspended in 200 μl cell culture media (cell concentration should be 500,000 cells per 1000) and 100 μl were transferred per well to a U-bottom 96 well cell culture plate. 100 μl of TRP2 or control peptides at a concentration of 2.5 μM in complete medium were added per well. After 2 h of incubation at 37° C., 5% CO₂, 50 μl GolgiPlug (BD) in dilution 1:200 was added per well.

Then, the incubation proceeded for an additional 6 h at 37° C. 1 μl Fc-block (BD) in 19 μl medium was added per well 20 minutes prior to cells were harvested by centrifugation at 400 g, 5 min at 4° C. Cells were washed once with 200 μl PBS-1% FBS, and stained with 1 μl FITC-conjugated anti-mouse CD8 antibody in 19 μl PBS-1% FBS. Staining proceeded for 30 min at 4° C. in the dark. Then, cells were washed twice with 200 μl PBS-1% FBS, and collected by centrifugation (400 g, 5 min, 4° C.). Cells were fixed in 100 μl of Cytofix/Cytoperm™ solution (BD) for 20 min at 4° C. in the dark, and then washed two times with 1×Perm/Wash™ solution (BD), before staining for interferon gamma (IFN-γ). 20 μl of Perm/Wash™ solution containing 1 μl anti-mouse IFN-γ PE-conjugated (BD) antibody was added to the cells for 30 min at 4° C. in the dark. Then, cells were washed 2 times with 1×Perm/Wash™ solution (1 ml/wash for staining in tubes) and resuspended in PBS-1% FBS prior to flow cytometry analysis.

Results are shown in FIG. 7. To investigate the role of type I interferon induction for pDAI adjuvant, mice deficient for the type I interferon receptor and interferon response factor 3 were vaccinated with pDAI and pTRP2. The adjuvant effect of DAI was lost in IFN-α/βR−/− indicating dependence on intact type I signalling for DAI adjuvant for this antigen. This was further underlined by the loss of adjuvant effect of DAI in the IRF3−/− mice, which knocks out one prominent pathway of DAI signalling leading to type I interferon production. (*P<0.05, Student's t-test). Error bars represent the standard error of the mean.

### Example 6

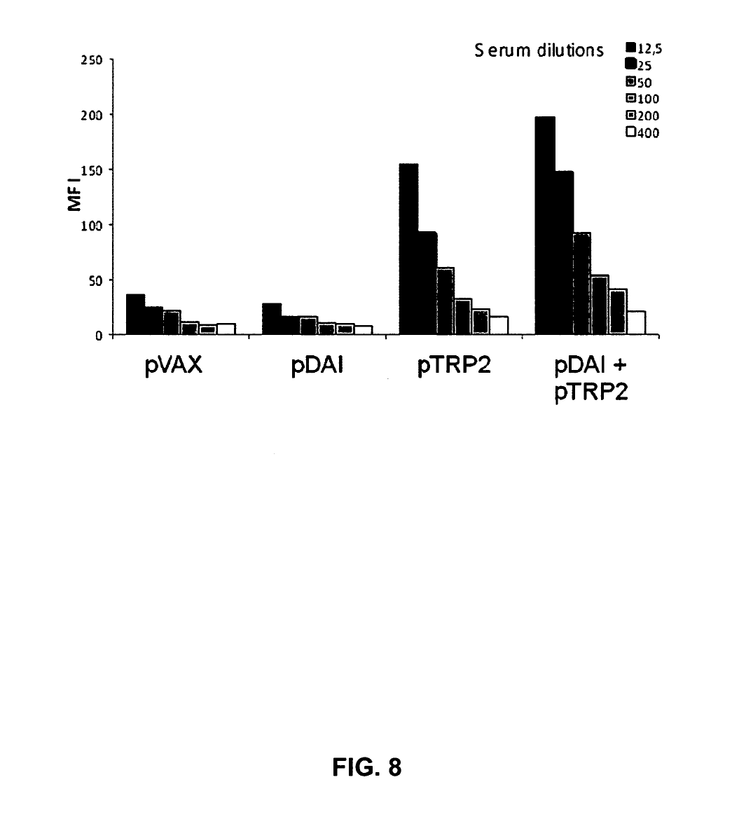

pDAI co-administration increases antibody responses to TRP2. Antibodies were bound to cells transfected with the TRP2 gene and detected with an anti-mouse anti-IgG FITC-conjugated antibody by flow cytometry analysis (FIG. 8).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 40 μg empty vector (pVAX), 20 μg DAI encoding vector plus 20 μg of empty vector (pDAI, n=9), 20 μg vector encoding human Tyrosine Related Protein 2 plus 20 μg empty vector (pTRP2, n=9), or 20 μg of a vector encoding DAI plus 20 μg pTRP2 vector (pDAI+pTRP2, n=9). 13 days after the second immunization, lymphocytes from peripheral blood were isolated as follows. Mice were bled from the tail and ˜300 μl blood was collected into tubes containing 100 μl heparin solution. Blood samples were centrifuged for 5 min, 350 g, and the plasma was collected. Samples were pooled and serially diluted in PBS-1% FBS two-fold steps between 12.5 and 400 fold. Four days before analysis, 2-3 million HEK293 cells were seeded in 10 cm dishes coated with 0.02% Gelatin for 15 min at 37° C. Cells were grown over night in DMEM 10% FBS without antibiotics. Cells were washed with PBS and 6 ml Opti-MEM was added.

Then, 24 μg pTRP2 was mixed with 1.5 ml Opti-MEM, and in parallel, 60 μl Lipofectamine 2000 was mixed with 1.5 ml Opti-MEM. The transfection mixes were incubated 5 min at room temperature before mixing and continued incubation for 20 min. The transfection mix was then dripped over the cell culture, and cells were left for 3 h at 37° C. with the transfection before addition of 9 ml of DMEM with 20% FBS. Medium was changed to DMEM 10% FBS after 3 h. cells were cultured for 48 h at 37° C. before they were harvested by pipetting up and down. Cells were washed twice with PBS, centrifuged at 400 g for 5 min and resuspended in PBS-1% FBS before they were plated in a cells in a 96 well plate. Cells were centrifuged for 2 min 350 g, and medium was discarded. 20 μl plasma sample in the various dilutions was added to the cells in different and binding occurred for 1 h at 37° C. Cells were washed twice with PBS-1% FBS, and 20 μl of the secondary antibody (fluorescent anti-mouse Immunoglobulins) in a 1:20 dilution was added. The detection antibody was incubated for 20 min at 4° C. before the cells were washed twice as above, and cells were resuspended in 200 μl 4% paraformaldehyde and fixed for 15 min at +4° C. Cells were again washed as above, and collected by centrifugation, 400 g for 5 min, washed twice in PBS-1% FBS and resuspended in 200 μl PBS-1% FBS and transferred to FACS tubes until flow cytometry analysis.

Results are shown in FIG. 8. In plasma samples from pDAI+pTRP2 co-immunized mice, there was a tendency to an increased response as compared to mice immunized with pTRP2 only. There was no reactivity in pDAI and pVAX immunized animals.

### Example 7

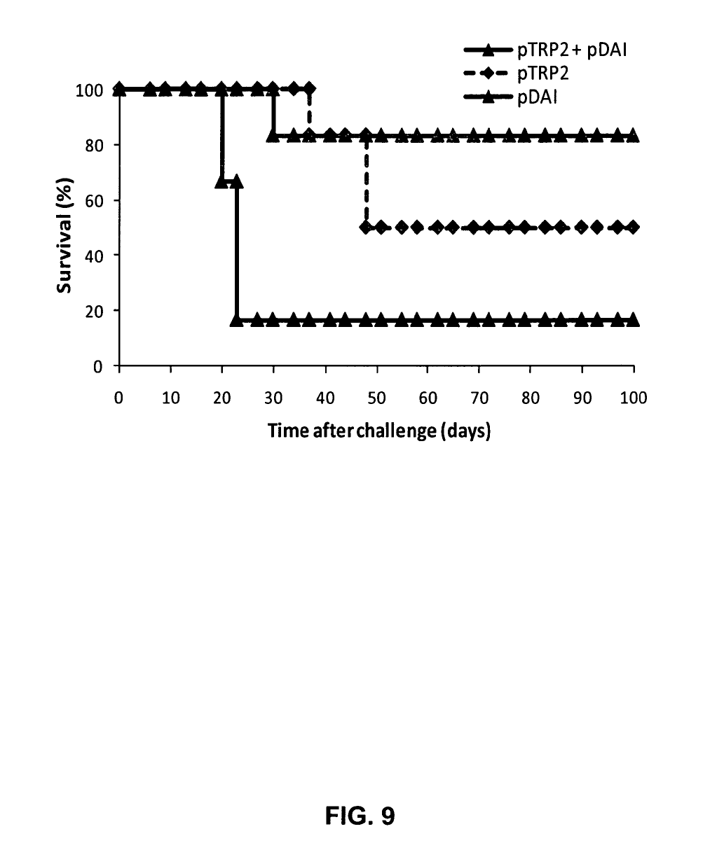

Mice co-immunized with pDAI and pTRP2 have improved survival to challenge with B16 tumor cells as compared to pTRP2 only or control animals (FIG. 9).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=6), 20 μg empty vector plus 20 μg vector encoding human Tyrosine Related Protein 2 (pTRP2, n=6), or 20 μg of a vector encoding DAI plus 20 μg vector encoding human Tyrosine Related Protein 2 (pDAI+pTRP2, n=11). Two weeks after the second immunization, mice were challenged with 50,000 B16 tumor cells s.c. in the flank. Tumor growth was monitored by animal facility staff twice a week by palpating and measuring tumor size with a caliper. Animals were sacrificed when tumor size exceeded 10×10×10 mm in size, and survival was plotted over time.

The results are shown in FIG. 9. The adjuvant effect of pDAI on pTRP2 seen in CD8+ T cells translated into a higher degree of protection (82% compared to 50% in the pTRP2 immunized group) to tumor challenge with an aggressive melanoma tumor cell line. Mice that stayed tumour free remained healthy throughout the experiment (more than 100 days post challenge).

### Example 8

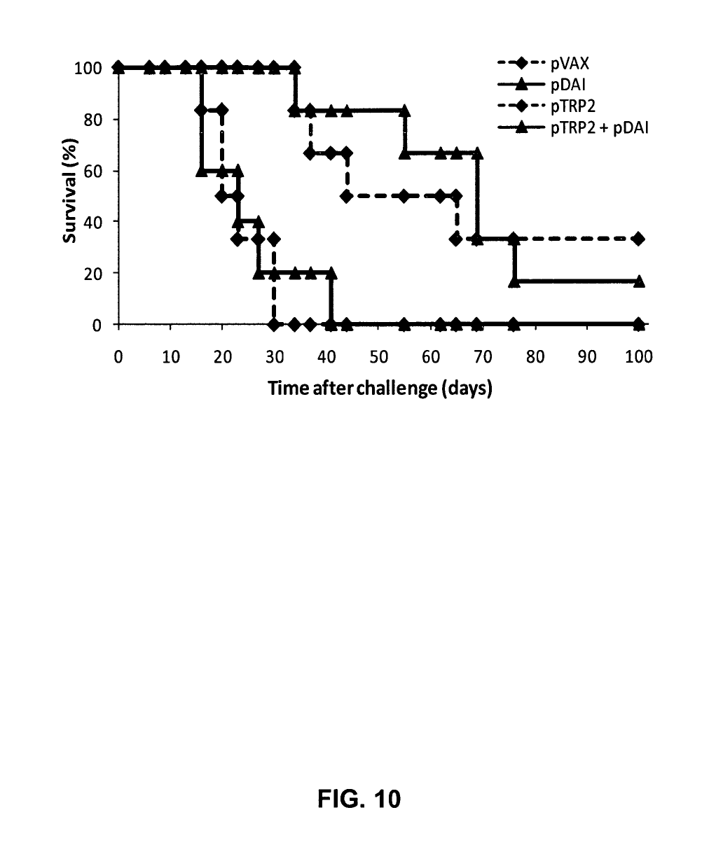

Improved survival of mice co-immunized with pDAI and pTRP2 as compared to pTRP2 is dependent of IRF3 (FIG. 10).

IRF3−/− mice (C57BL/6 background) mice were anesthetized with isoflurane and immunized two times, 2 weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 40 μg of empty vector (pVAX, n=6), 20 μg DAI encoding vector plus 20 μg of empty vector (pDAI, n=5), 20 μg vector encoding human Tyrosine Related Protein 2 plus 20 μg empty vector (pTRP2, n=6), or 20 μg of a vector encoding DAI plus 20 μg empty vector (pDAI+pTRP2, n=6). Two weeks after the second immunization, mice were challenged with 50,000 B16 tumor cells s.c. in the flank. Tumor growth was monitored by animal facility staff twice weekly by palpating and measuring tumor size with a caliper. Animals were sacrificed when tumor size exceeded 10×10×10 mm in size, and survival was plotted over time.

The adjuvant effect of pDAI on tumor protection seen on wild type C57BL6 was not observed in IRF3−/− mice challenged with the aggressive B16 melanoma cells (FIG. 10). Mice that stayed tumour free remained healthy throughout the experiment (more than 100 days post challenge).

### Example 9

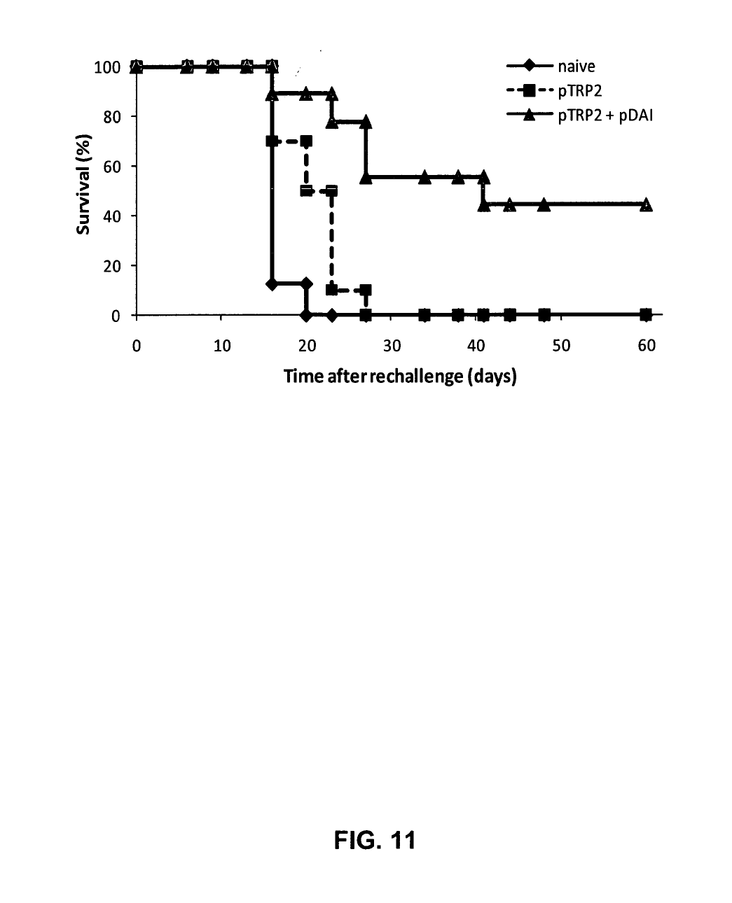

pDAI+pTRP2 immunization induces long-term protection to rechallenge (FIG. 11).

DNA in PBS was injected intradermally two times, two weeks apart at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg vector encoding human Tyrosine Related Protein 2 plus 20 μg empty vector (pTRP2), or 20 μg of a vector encoding DAI plus 20 μg pTRP2pTRP2 vector (pDAI+pTRP2). Two weeks after the second immunization, mice were challenged with 50,000 B16 tumor cells s.c. in the flank. Tumor growth was monitored by animal facility staff twice a week by palpating and measuring tumor size with a caliper. Animals were sacrificed when tumor size exceeded 10×10×10 mm in size, and survival was plotted over time. Mice surviving an initial challenge with 50,000 BI 6 tumor cells were collected from groups immunized with pTRP2 (n=10) and pTRP2+ pDAI (n=9). These mice, together with naïve controls (n=8) were re-challenged with a high dose of B16 tumor cells (150,000) in the opposite side (right flank) three months after the initial challenge. Tumor growth was monitored by animal facility staff twice a week by palpating and measuring tumor size with a caliper. Animals were sacrificed when tumor size exceeded 10×10×10 mm in size, and survival was plotted over time.

FIG. 11 shows that the adjuvant effect of pDAI on pTRP2 vaccination seen in CD8+ T cells translated into a higher degree of protection at 3 months after the first challenge to tumor challenge with a high dose of an aggressive melanoma tumor cell line. Tumor protection was 44% in pDAI+pTRP2 immunized mice as compared to 0% in pTRP2 immunized and naïve mice. Mice that stayed tumour free remained healthy throughout the experiment (60 days post rechallenge).

### Example 10

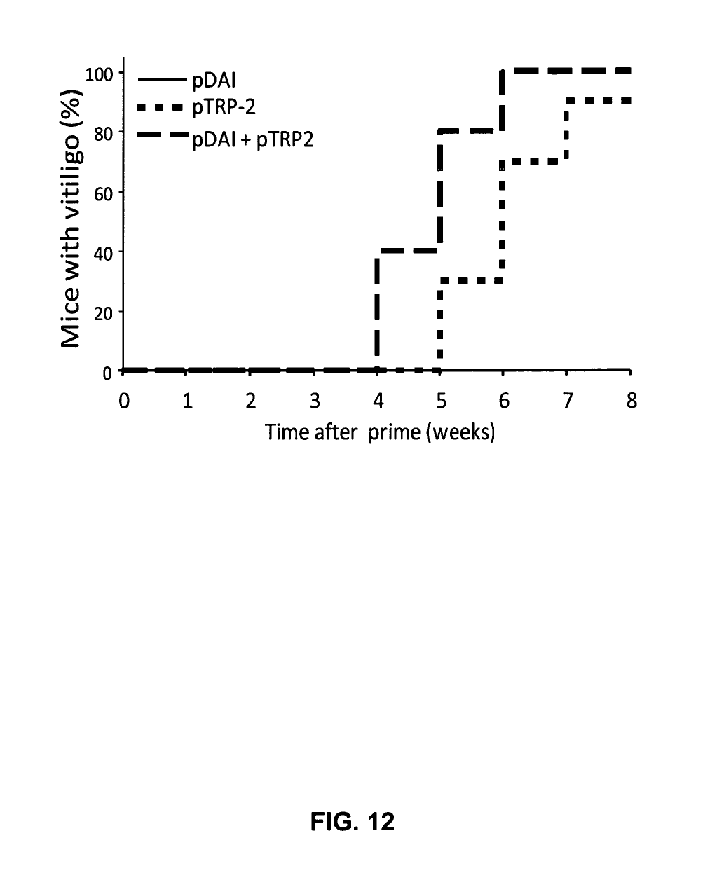

Emergence of autoimmune vitiligo is faster after immunization with pTRP2 when mice are co-immunized with pDAI (FIG. 12).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=9), 20 μg vector encoding human Tyrosine Related Protein 2 plus 20 μg empty vector (pTRP2, n=9), or 20 μg of a vector encoding DAI plus 20 μg pTRP2pTRP2 vector (pDAI+pTRP2, n=9).

FIG. 12 shows that after the second immunization, vitiligo emerged on most TRP2 immunized animals. Vitiligo is a CD8+ T cell mediated autoimmune response to melanin producing cells. Melanin is produced by cells expressing murine TRP2, and killing of these cells results in vitiligo. Initially, vitiligo is localized around the sites of immunization, but over time, the vitiligo spreads over the entire body of the mouse. Here, we plotted the appearance of vitiligo in mice vaccinated with pTRP2 with or without DAI. In mice co-immunized with DAI, vitiligo appeared sooner after the second immunization, which could be a sign of a faster kinetics of the CD8+ T cell response or a higher magnitude of the CD8+ T cell response in DAI immunized mice.

### Example 11

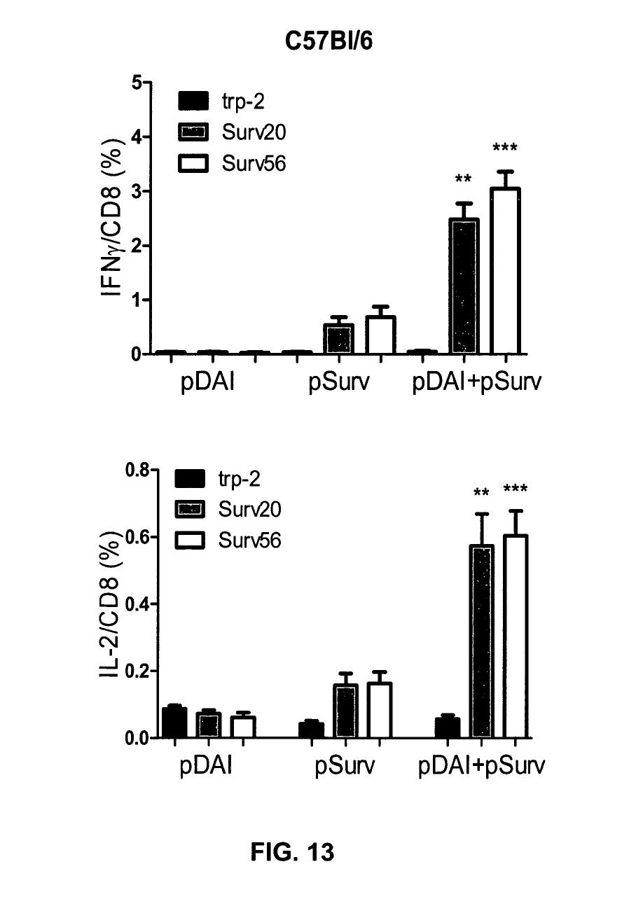

Mice co-immunized with pDAI and survivin encoding plasmid (pSURV) have a stronger T cell response than mice immunized with pSURV alone (FIG. 13).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=7), 20 μg vector encoding human Survivin plus 20 μg of empty vector (pSurv, n=8) or 20 μg of pDAI+20 μg vector pSury (pDAI+pSurv, n=8).). 12 days after the second immunization, lymphocytes from peripheral blood were isolated as follows. Mice were bled from the tail and ˜300 μl blood was collected into tubes containing 100 μl heparin solution. Erythrocytes were lysed by adding 1 ml Ammonium Chloride (ACK) Buffer (1×Pharm Lyse BD) for 5 min a room temperature. 10-12 ml of media was added and samples were centrifuged at 400 g for 5 min. The supernatant was discarded, and the cell pellet was resuspended in 7 ml complete cell culture medium (RPMI1640, 10% FBS, Sodium pyruvate, penicillin/streptomycin and Non-essential amino acids) and centrifuged at 400 g for 5′ min. The cell pellet was resuspended in 300 μl cell culture media and 100 μl were transferred per well to a U-bottom 96 well cell culture plate. 100 μl of surviving specific peptides Surv20, Surv56 or control peptide at a concentration of 2.5 μM in complete medium were added per well. After 2 h of incubation at 37° C., 5% CO₂, 50 μl GolgiPlug (BD) in dilution 1:200 was added to each well.

Then, the incubation proceeded for an additional 6 h at 37° C. 1 μl Fc-block (BD) in 19 μl medium was added per well 20 minutes prior to cells were harvested by centrifugation at 400 g, 5 min at 4° C. Cells were washed once with 200 μl PBS-1% FBS, and stained with 1 μl PE-conjugated anti-mouse CD8 antibody in 19 μl PBS-1% FBS. Staining proceeded for 30 min at 4° C. in the dark. Then, cells were washed twice with 200 μl PBS-1% FBS, and collected by centrifugation (400 g, 5 min, 4° C.). Cells were fixed in 100 μl of Cytofix/Cytoperm™ solution (BD) for 20 min at 4° C. in the dark, and then washed two times with 1×Perm/Wash™ solution (BD), before staining for interferon gamma (IFN-γ). 20 of Perm/Wash™ solution containing 1 μl anti-mouse IFN-γ FITC-conjugated (BD) antibody and 1 μl anti-mouse IL-2 APC-conjugated (BD) was added to the cells for 30 min at 4° C. in the dark. Then, cells were washed 2 times with 1×Perm/Wash™ solution (1 ml/wash for staining in tubes) and resuspended in PBS-1% FBS prior to flow cytometry analysis.

In mice immunized with pDAI, CD8+ T cells showed only background IFN-γ production upon stimulation with the two survivin-derived peptides, whereas mice immunized with the pSURV plasmid responded with IFN-γ production in about 1% of the CD8+ T cells (FIG. 13). When mice were co-immunized with pSURV and pDAI, this proportion was significantly increased to about 6% (p<0.002 for both peptides, Mann-Whitney U-test). The IL-2 response was lower in both groups, but was significantly increased in the pDAI+pSURV group (p<0.003 for both peptides, Mann-Whitney U-test). One representative of two independent experiments is shown. Error bars represent the standard error of the mean.

### Example 12

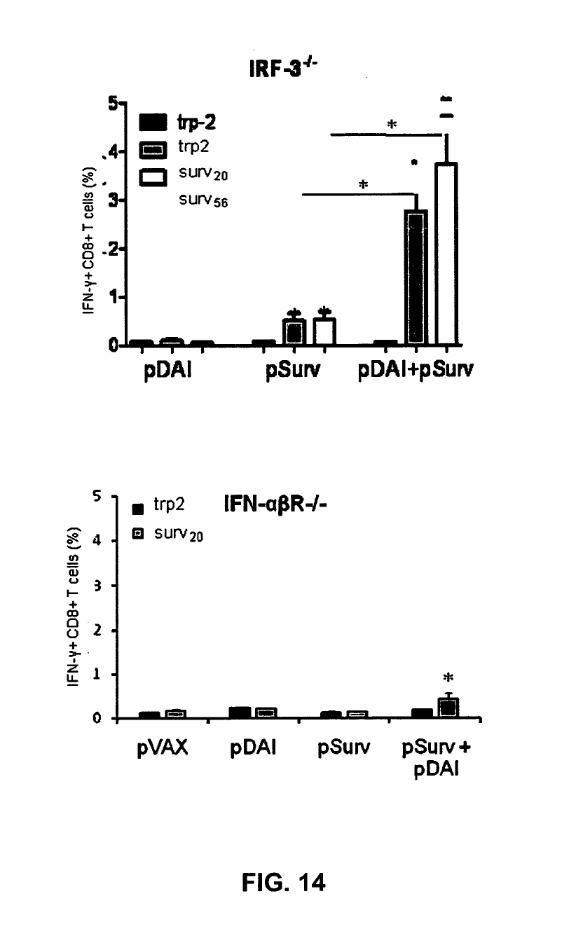

The adjuvant effect of pDAI to pSURV is not mediated by type I interferons (FIG. 14).

IFN-α/βR−/− mice (n=7 per group) and IRF3−/− mice (n=8 per group) were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI), 20 μg vector encoding human Survivin plus 20 μg empty vector (pSurv) or 20 μg of pDAI+20 μg vector pSury (pDAI+pSurv) 12 days after the second immunization, lymphocytes from peripheral blood were isolated as follows. Mice were bled from the tail and ˜300 μl blood was collected into tubes containing 100 μl heparin solution. Erythrocytes were lysed by adding 1 ml Ammonium Chloride (ACK) Buffer (1×Pharm Lyse BD) for 5 min a room temperature. 10-12 ml of media was added and samples were centrifuged at 400 g for 5 min. The supernatant was discarded, and the cell pellet was resuspended in 7 ml complete cell culture medium (RPMI1640, 10% FBS, Sodium pyruvate, penicillin/streptomycin and Non-essential amino acids) and centrifuged at 400 g for 5 min. The cell pellet was resuspended in 300 μl cell culture media and 100 μl were transferred per well to a U-bottom 96 well cell culture plate. 100 μl of surviving specific peptides Surv20, Surv56 or control peptide at a concentration of 2.5 μM in complete medium were added per well. After 2 h of incubation at 37° C., 5% CO₂, 50 μl GolgiPlug (BD) in dilution 1:200 was added to each well.

Then, the incubation proceeded for an additional 6 h at 37° C. 1 μl Fc-block (BD) in 19 μl medium was added per well 20 minutes prior to cells were harvested by centrifugation at 400 g, 5 min at 4° C. Cells were washed once with 200 μl PBS-1% FBS, and stained with 1 μl PE-conjugated anti-mouse CD8 antibody in 19 μl PBS-1% FBS. Staining proceeded for 30 min at 4° C. in the dark. Then, cells were washed twice with 200 μl PBS-1% FBS, and collected by centrifugation (400 g, 5 min, 4° C.). Cells were fixed in 100 μl of Cytofix/Cytoperm™ solution (BD) for 20 min at 4° C. in the dark, and then washed two times with 1×Perm/Wash™ solution (BD), before staining for interferon gamma (IFN-γ). 20 μl of Perm/Wash™ solution containing 1 μl anti-mouse IFN-γ FITC-conjugated (BD) antibody and 1 μl anti-mouse IL-2 APC-conjugated (BD) was added to the cells for 30 min at 4° C. in the dark. Then, cells were washed 2 times with 1×Perm/Wash™ solution (1 ml/wash for staining in tubes) and resuspended in PBS-1% FBS prior to flow cytometry analysis.

To investigate the role, of type I interferon induction on pDAI adjuvanticity, we immunized mice deficient for the type I interferon receptor and interferon response factor 3 with pDAI and pSurv. The overall responses were low in the IFN-α/βR−/− mice but there was still a small but significant adjuvant effect of DAI (p<0.05, Student's T-test) (FIG. 14). This result was further emphasized in the IRF3−/− mice, in which the adjuvant effect was intact and significant (p≦0.01). For this antigen, the adjuvant effect of DAI thus seems to be independent of the type I interferon response. Error bars represent the standard error of the mean.

### Example 13

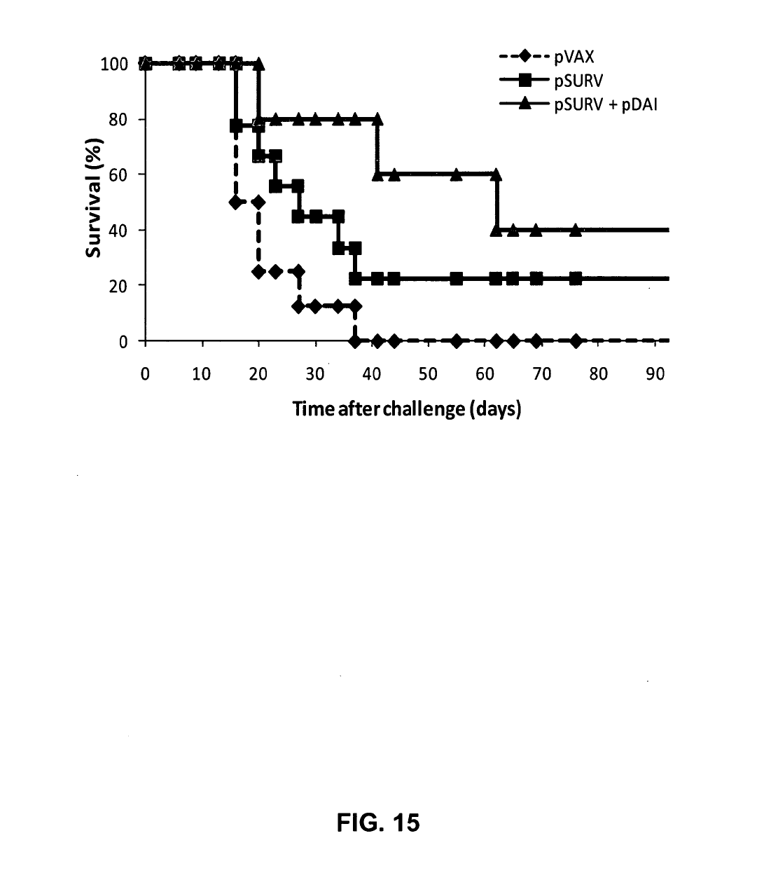

Mice co-immunized with pDAI and pSURV have improved survival to challenge with B16 tumor cells as compared to pSURV only or control animals (FIG. 15).

C57BL/6 mice were anesthetized with isoflurane and immunized two times, two weeks apart with 40 μg plasmid DNA in PBS injected intradermally at two sites (20 μg each) near the base of the tail using a 29-gauge insulin-grade syringe (Micro-Fine U-100, BD Consumer Healthcare, Franklin Lakes, N.J., USA). Immediately, a parallel needle array electrode (two rows of four 2-mm pins, 1.5×4 mm gaps) was placed over the injection blebs, and electric pulses (two 1125 V/cm pulses followed by eight 275 V/cm pulses) were applied using Derma Vax™ Intradermal Delivery System (Cyto Pulse Sciences, Inc). Mice were injected with 20 μg DAI encoding vector plus 20 μg of empty vector (pDAI, n=8), 20 μg empty vector plus 20 μg vector encoding human survivin (pSURV, n=9), or 20 μg of a vector encoding DAI plus 20 μg vector encoding human survivin (pDAI+pSURV, n=5). One week after the second immunization, mice were challenged with 100,000 B16 tumor cells s.c. in the flank. Tumor growth was monitored by animal facility staff twice a week by palpating and measuring tumor size with a caliper. Animals were sacrificed when tumor size exceeded 10×10×10 mm in size, and survival was plotted over time.

The adjuvant effect of pDAI on pSURV seen in CD8+ T cells translated into a higher degree of protection (40% compared to 22% in the pSURV only immunized group) to tumor challenge with an aggressive melanoma tumor cell line (FIG. 15). Mice that stayed tumour free remained healthy throughout the experiment (more than 100 days post challenge).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed is:

1. A vaccine composition, comprising:
a vaccine antigen, and
an activator of interferon regulatory factors, in an amount that is in addition to existing cellular levels of the activator.

2. The vaccine composition of claim 1 wherein said vaccine antigen is selected from the group consisting of polypeptides, proteins, polysaccharides, haptens, glycosylated proteins, lipoproteins, lipopolysaccharides, cells, cell extracts, polysaccharide conjugates, lipids, glycolipids and carbohydrates.

3. The vaccine composition of claim 1 wherein said vaccine antigen is selected from the group consisting of:
a polynucleotide vector which when expressed produces a polypeptide, and
a polynucleotide vector which when expressed produces a protein.

4. The vaccine composition of claim 1 wherein said activator of interferon regulatory factors is selected from the group consisting of polypeptides and proteins.

5. The vaccine composition of claim 1 wherein said activator of interferon regulatory factors is selected from the group consisting of DAI, RIG-1, and Mda-5.

6. The vaccine composition of claim 1 wherein said activator of interferon regulatory factors is selected from the group consisting of:
a polynucleotide vector which when expressed produces a polypeptide, and
a polynucleotide vector which when expressed produces a protein.

7. The vaccine composition of claim 6 wherein said polynucleotide which when expressed produces a protein is selected from the group consisting of DAI, RIG-1, and Mda-5.

8. A method for vaccinating, comprising the steps of:
administering a vaccine antigen, and
administering an activator of interferon regulatory factors.

9. The method for vaccinating of claim 8 wherein the vaccine antigen and the activator of interferon regulatory factors are administered simultaneously.

10. The method for vaccinating of claim 8 wherein the vaccine antigen and the activator of interferon regulatory factors are administered at different times.

11. A vaccine adjuvant composition, comprising:
a polynucleotide vector which is capable of expressing a polynucleotide binding protein which leads to the production of immunomodulators, and
a pharmaceutically acceptable carrier.

12. A vaccine composition, comprising:
a polynucleotide vector capable of expressing a vaccine antigen, and
a polynucleotide vector capable of expressing an activator of interferon regulatory factors, in an amount that is in addition to existing cellular levels of the activator.


SEQUENCE LISTING (22 sequences)

SEQ ID NO: 1 - DNA, 20 bp, enterobacteria phage T7
taatacgactcactataggg

SEQ ID NO: 2 - DNA, 18 bp, Bos primigenius
tagaaggcacagtcgagg

SEQ ID NO: 3 - DNA, 23 bp, Mus musculus
atggctagrctctgtgctttcct

SEQ ID NO: 4 - DNA, 24 bp, Mus musculus
agggctctccagayttctgctctg

SEQ ID NO: 5 - DNA, 21 bp, Mus musculus
catcaactataagcagctcca

SEQ ID NO: 6 - DNA, 21 bp, Mus musculus
ttcaagtggagagcagttgag

SEQ ID NO: 7 - DNA, 20 bp, Mus musculus
gctttaacagcaggccagac

SEQ ID NO: 8 - DNA, 20 bp, Mus musculus
gcaagcaccaggtgtcaagt

SEQ ID NO: 9 - DNA, 22 bp, Mus musculus
ccccagaagaccctcctgatag

SEQ ID NO: 10 - DNA, 22 bp, Mus musculus
ccgaaggtaaggctgttgtttg

SEQ ID NO: 11 - DNA, 21 bp, Mus musculus
tgcaggcgaccaggagagaca

SEQ ID NO: 12 - DNA, 21 bp, Mus musculus
gaagctgggcacctcaaagat

SEQ ID NO: 13 - DNA, 24 bp, Mus musculus
gatgctaccaaactggatataatc

SEQ ID NO: 14 - DNA, 23 bp, Mus musculus
ggtccttagccactccttctgtg

SEQ ID NO: 15 - DNA, 23 bp, Mus musculus
gtgaaaataagagcaaggcagtg

SEQ ID NO: 16 - DNA, 21 bp, Mus musculus
attcatggtcttgtagacacc

SEQ ID NO: 17 - DNA, 20 bp, Mus musculus
agtaccctgtgccttggtag

SEQ ID NO: 18 - DNA, 17 bp, Mus musculus
gattctgaactgcgttg

SEQ ID NO: 19 - DNA, 22 bp, Mus musculus
ctacaggcttgtcactcgaatt

SEQ ID NO: 20 - DNA, 21 bp, Mus musculus
aatggcctccctctcatcagt

SEQ ID NO: 21 - DNA, 36 bp, Mus musculus
agtcgaattcccaccatggcagaagctcctgttgac

SEQ ID NO: 22 - DNA, 33 bp, Mus musculus
agtcgcggccgctcattgcttgctcagtcctgt